(19) United States
(12) Patent Application Publication
(10) Pub. No.: US 2018/0267046 A1
(43) Pub. Date: Sep. 20, 2018

(54) METHODS FOR DIAGNOSING AND TREATING CANCER

(71) Applicant: MiCareo Taiwan Co., Ltd., Taipei City (TW)

(72) Inventors: Ju-Yu Tseng, Taipei City (TW); Yen-Ru Chen, Taipei City (TW); Chia-Ying Lee, Taipei City (TW); Li-Fan Wu, Taipei City (TW); Shin-Hang Wang, Taipei City (TW); Jui-Lin Chen, Taipei City (TW); Chwen-Cheng Chen, Taipei City (TW)

(73) Assignee: MiCareo Taiwan Co., Ltd., Taipei City (TW)

(21) Appl. No.: 15/919,734

(22) Filed: Mar. 13, 2018

Related U.S. Application Data
(60) Provisional application No. 62/471,083, filed on Mar. 14, 2017.
(60) Provisional application No. 62/584,634, filed on Nov. 10, 2017.

(51) Int. Cl. G01N 33/574; G01N 33/49

(52) U.S. Cl. CPC G01N 33/57446; G01N 2033/57465; G01N 33/492; G01N 33/57419

(57) ABSTRACT

Disclosed is a method for identifying a subject at risk of developing a recurrent or metastatic cancer, comprising detecting PD-L1⁺ circulating tumor cells in a blood sample, a tissue fluid sample or a specimen of the subject. Also disclosed is method for treating a cancer comprising identifying a subject having one ore more PD-L1⁺ circulating tumor cells by detecting PD-L1⁺ circulating tumor cells in a blood sample, a tissue fluid sample or a specimen of the subject, and administering a treatment to the subject.

### CROSS REFERENCE TO RELATED APPLICATIONS

This non-provisional application claims the benefit under 35 U.S.C. § 119(e) to U.S. Provisional Application No. 62/471,083, filed on Mar. 14, 2017, and to U.S. Provisional Application No. 62/584,634, filed on Nov. 10, 2017, all of which are hereby expressly incorporated by reference into the present application.

### FIELD OF THE INVENTION

The present invention pertains to methods for diagnosing and treating cancer.

### BACKGROUND OF THE INVENTION

Circulating tumor cells (CTCs) presence in circulation play active roles in mediating metastasis[1]. Enumeration of CTCs was reported as a prognostic predictor for metastatic colorectal cancer (mCRC) patients [2]. In most of the previous studies, the number of CTCs was enumerated from blood drawn by venipuncture of the forearm [3, 4]. It was suggested that the presence of circulating tumor cells expressing PD-L1 in non-small cell lung cancer patients at 6 months after the treatment of Nivolumab (PD-1 inhibitor) corresponds to a therapy escape or poor prognosis [5].

Until now, there is no promising method or kit for early detection or diagnosis of a metastatic cancer.

### BRIEF SUMMARY OF THE INVENTION

The present invention is based on the unexpected finding that in patients having a gastrointestinal cancer or head and neck squamous-cell carcinoma, the presence/level of circulating tumor cells expressing PD-L1 (called as “PD-L1⁺ circulating tumor cells”) before a therapy for, or during a surgery of curative resection of the gastrointestinal cancer or head and neck squamous-cell carcinoma, correlates with the metastasis of cancers or the prognosis of patients.

Accordingly, in one aspect, the present invention provides a method for identifying a subject at risk of developing a recurrent or metastatic cancer, comprising detecting PD-L1⁺ circulating tumor cells in a blood sample, a tissue fluid sample or a specimen of the subject, wherein the presence of one or more PD-L1⁺ circulating tumor cells indicates that the subject is at risk of developing a recurrent or metastatic cancer, and wherein the subject has a gastrointestinal cancer or head and neck squamous-cell carcinoma and the blood sample, the tissue fluid sample or the specimen is derived from the subject before a therapy for, or during a surgery of curative resection of the gastrointestinal cancer or head and neck squamous-cell carcinoma.

In one embodiment of the present invention, the cancer is a gastrointestinal cancer, particularly a colorectal cancer. In another embodiment, the cancer is head and neck squamous-cell carcinoma.

In another aspect, the present invention provides a method for treating a cancer comprising: (a) identifying a subject having one or more PD-L1⁺ circulating tumor cells by detecting PD-L1⁺ circulating tumor cells in a blood sample, a tissue fluid sample or a specimen of the subject, wherein the subject has a gastrointestinal cancer or head and neck squamous-cell carcinoma and the blood sample, the tissue fluid sample or the specimen is derived from the subject before a therapy for, or during a surgery of curative resection of the gastrointestinal cancer or head and neck squamous-cell carcinoma, and (b) administering a treatment to the subject.

In a further aspect, present invention provides a method for predicting prognosis of a patient having gastrointestinal cancer or head and neck squamous-cell carcinoma, comprising detecting PD-L1⁺ circulating tumor cells in a blood sample, a tissue fluid sample or a specimen of the patient, wherein the presence of one or more PD-L1⁺ circulating tumor cells indicates poor prognosis or overall survival of the patient, wherein the blood sample, the tissue fluid sample or the specimen is derived from the patient before a therapy for, or during a surgery of curative resection of the gastrointestinal cancer or head and neck squamous-cell carcinoma.

According to certain preferred embodiments of the present invention, said treatment is an immunotherapy. In some other embodiments, the subject is administered with an immunotherapy, in combination with a chemotherapy or a targeted therapy.

According to certain embodiments of the present invention, the cancer is a recurrent or metastatic cancer.

In one embodiment of the present invention, the cancer is a gastrointestinal cancer, particularly a colorectal cancer. In another embodiment, the cancer is head and neck squamous-cell carcinoma.

It is to be understood that both the foregoing general description and the following detailed description are exemplary and explanatory only and are not restrictive of the invention.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred.

The patent or application file contains at least one color drawing. Copies of this patent or patent application publication with color drawing will be provided by the USPTO upon request and payment of the necessary fee.

In the drawings:

FIG. 1 illustrates the sample preparation for circulating tumor cell (CTC) enumeration via MiSelect R System.

FIG. 2 shows typical CTC images.

FIG. 3 shows the comparison of CTC counts in peripheral blood (PB) and mesenteric venous blood (MVB).

FIG. 4A shows the results on CTC counts in PB of colorectal cancer (CRC) patients at various stages. FIG. 4B shows the results on CTC counts in MVB of CRC patients at various stages.

FIG. 5 shows the heterogeneity of PD-L1 expressions on CTC.

FIG. 6 shows PD-L1⁺ CTC counts in PB and MVB.

FIG. 7 shows PD-L1⁺ CTC counts in MVB at various stages.

FIG. 8 shows the frequency of occurrence of PD-L1⁺ CTC at various stages.

FIG. 9 shows the heterogeneity of PD-L1 expressions on CTC

### DETAILED DESCRIPTION OF THE INVENTION

In one aspect, the present invention provides a method for identifying a subject at risk of developing a recurrent or metastatic cancer, comprising detecting PD-L1⁺ circulating tumor cells in a blood sample, a tissue fluid sample or a specimen of the subject, wherein the presence of PD-L1⁺ circulating tumor cells indicates that the subject is at risk of developing a recurrent or metastatic cancer, and wherein the subject has a gastrointestinal cancer or head and neck squamous-cell carcinoma and the blood sample, the tissue fluid sample or the specimen is derived from the subject before a therapy for, or during a surgery of curative resection of the gastrointestinal cancer or head and neck squamous-cell carcinoma.

According to the present invention, a higher level of the PD-L1⁺ circulating tumor cells indicates a higher risk of developing a recurrent or metastatic cancer.

In one embodiment of the present invention, the cancer is a gastrointestinal cancer, particularly a colorectal cancer. For gastrointestinal cancers, the blood sample is preferably a mesenteric venous blood sample. The mesenteric venous blood sample may be derived during surgeries of curative resection of the gastrointestinal cancer. The gastrointestinal cancer includes but is not limited an esophageal cancer, a gastric cancer, a gastrointestinal stromal tumor, a pancreatic cancer, a liver cancer, a gallbladder cancer, a colorectal cancer, and an anal cancer. In a particular example of the present invention, the cancer is a colorectal cancer.

In another embodiment, the cancer is head and neck squamous-cell carcinoma.

In another aspect, the present invention provides a method for treating a cancer comprising: (a) identifying a subject having PD-L1⁺ circulating tumor cells by detecting PD-L1⁺ circulating tumor cells in a blood sample, a tissue fluid sample or a specimen of the subject, wherein the subject has a gastrointestinal cancer or head and neck squamous-cell carcinoma and the blood sample, the tissue fluid sample or the specimen is derived from the subject before a therapy for, or during a surgery of curative resection of the gastrointestinal cancer or head and neck squamous-cell carcinoma, and (b) administering a treatment to the subject.

In a further aspect, present invention provides a method for predicting prognosis of a patient having a gastrointestinal cancer or head and neck squamous-cell carcinoma, comprising detecting PD-L1⁺ circulating tumor cells in a blood sample, a tissue fluid sample or a specimen of the patient, wherein the presence of one or more PD-L1⁺ circulating tumor cells indicates poor prognosis or overall survival of the patient, and wherein the blood sample, the tissue fluid sample or the specimen is derived from the patient before a therapy for, or during a surgery of curative resection of the gastrointestinal cancer or head and neck squamous-cell carcinoma.

According to the present invention, a higher level of the PD-L1⁺ circulating tumor cells indicates a worse prognosis of the patient.

The treatment includes but is not limited to immunotherapy, chemotherapy, radiation therapy, hormone therapy, or a combination thereof.

According to certain preferred embodiments of the present invention, said treatment is an immunotherapy. In some other embodiments, the subject is administered with an immunotherapy, in combination with a chemotherapy or a targeted therapy.

According to certain embodiments of the present invention, the cancer is a recurrent or metastatic cancer.

In one embodiment of the present invention, the cancer is a gastrointestinal cancer, particularly a colorectal cancer. In another embodiment, the cancer is head and neck squamous-cell carcinoma.

For gastrointestinal cancers, the blood sample is preferably a mesenteric venous blood sample. The mesenteric venous blood sample may be derived during surgeries of curative resection of the gastrointestinal cancer.

In certain embodiments of the present invention, the cancer is a gastrointestinal cancer, which includes, but is not limited to, an esophageal cancer, a gastric cancer, a gastrointestinal stromal tumor, a pancreatic cancer, a liver cancer, a gallbladder cancer, a colorectal cancer, and an anal cancer. In a particular example of the present invention, the cancer is a colorectal cancer.

In some other embodiments of the present invention, the cancer is head and neck squamous-cell carcinoma.

The present invention is further illustrated by the following examples, which are provided for the purpose of demonstration rather than limitation.

### Examples

Materials and Methods

1. Patient Characteristics

A total of 116 patients who underwent curative surgical resection at Taipei Veterans General Hospital between April 2016 and September 2017 were enrolled. A total 26 HNSCC, 9 HCC, 5 UC and 2 RCC patients who underwent receiving immunotherapy at Taipei Veterans General Hospital between October 2016 and September 2017 were also enrolled. The enrollment procedures followed the protocols approved by the Internal Review Board of Taipei Veterans General Hospital. All patients provided written informed consent. Patients who prior to colonoscopy examination or suspected of having colorectal cancer (CRC) with unconfirmed clinical stages were recruited. Six patients who had histological diagnosed tubular adenoma were also enrolled. The average (±SD) age of the assessable patients was 63.6±12.5 years (median, 64) (see Table 1 below). Primary tumor staging was confirmed by histologic examination of the resected primary tumor. Based on histologic examination, the subjects consisted of 116 CRC patients (24 stage I, 38 stage II, 42 stage III and 12 stage IV, respectively).

TABLE 1
Clinicopathological characteristics of the study population

| | Stage I | Stage II | Stage III | Stage IV |
|---|---|---|---|---|
| | (n = 24) | (n = 38) | (n = 42) | (n = 12) |
| Age | 64 | 68 | 64 | 59 |
| Median (range) | (37-79) | (38-92) | (37-84) | (47-90) |
| Gender | | | | |
| Male | 10 | 28 | 22 | 8 |
| Female | 14 | 10 | 20 | 4 |
| Tumor location | | | | |
| Colon | 20 | 27 | 29 | 9 |
| Rectum | 4 | 11 | 13 | 3 |
| T stage | | | | |
| T1 | 14 | 0 | 3 | 0 |
| T2 | 10 | 0 | 5 | 1 |
| T3 | 0 | 24 | 21 | 4 |
| T4 | 0 | 14 | 13 | 7 |
| N stage | | | | |
| N0 | 24 | 38 | 0 | 2 |
| N1 | 0 | 0 | 31 | 4 |
| N2 | 0 | 0 | 11 | 6 |
| CEA (≥5 ng/mL) | 3 | 17 | 17 | 10 |
| CA 19-9 (≥37 ng/mL) | 1 | 7 | 8 | 5 |

2. Blood Sample Collection

Blood samples for CTC analysis were obtained from CRC patients before curative resection of tumor. Sampling of blood from the antecubital veins of patients with CRC was conducted before surgery. During surgery, mesenteric venous blood samples were drawn from the main drainage vein of the diseased segment of the colon, for example, the inferior mesenteric vein for cancer of the sigmoid colon or rectum or the ileocolic vein if the tumor was located on the right side of the colon. To minimize the possibility of releasing CTCs by mechanical manipulation, colonoscopy was scheduled at least 1 day before the surgery. The surgical approach sought vascular control first, that is, ligation of the feeding artery at the beginning, followed by mesenteric vein cannulation and blood drawing. The tumor was left untouched until late in the surgery. Peripheral blood (PB) and mesenteric venous blood (MVB) samples for CTC analysis were obtained from 116 patients. Blood samples for CTC analysis were also obtained from other cancer patients before drug administrations.

3. CTC Enumeration and PD-L1 Expression Test on CTC

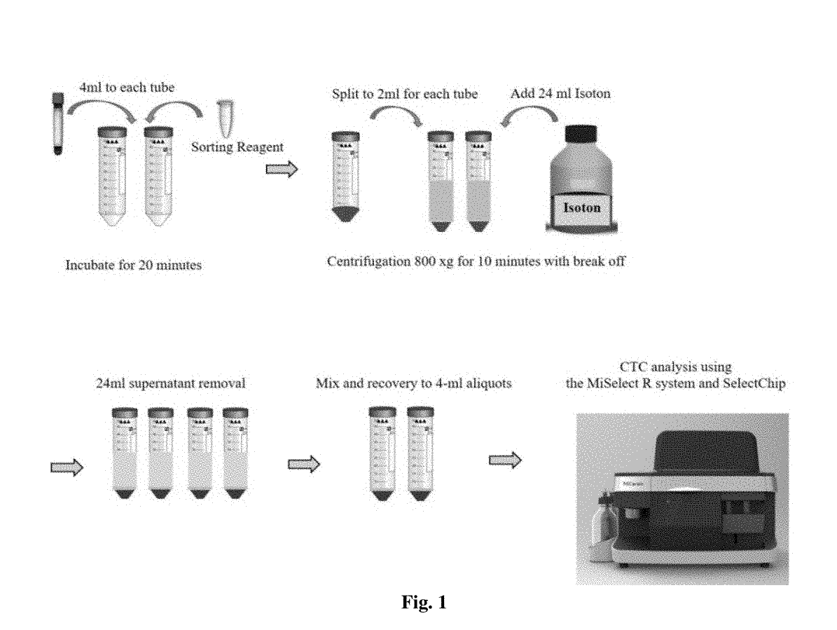

The sample preparation is shown in FIG. 1. Transfer two 4-ml aliquots of blood from K₂EDTA tube into two correspondingly labeled 50 ml conical centrifuge tubes. Samples incubate with Sorting Reagent (PE-conjugated anti-EpCAM antibody) of SelectKit for 20 minutes at room temperature. After staining, spilt each 4 ml blood to two 2-ml aliquots into two correspondingly labeled 50 ml conical centrifuge tubes, add 24 ml ISOTON Diluent into each tube. Centrifuge the sample at 800×g for a full 10 minutes with the brake off using a swing bucket centrifuge at room temperature. Following centrifugation, remove 24 ml supernatant of each tube and mix the samples following recovery of two 2-ml aliquots into 4 ml aliquots for CTC analysis. Process on the MiSelect R System within 1 hour of sample preparation.

MiSelect R System with SelectChip Dual (MiCareo Taiwan Co., Ltd) can sort and enrich CTC. Once the aliquots containing CTCs have been collected in SelectChip, blood cells, especially RBCs, are removed from the CTCs by an on-chip filtration system. After enrichment of CTCs, Fixation and Staining Reagent of SelectKit are automated added for identification and enumeration of CTCs. Anti-panCK APC is specific targeting for the intracellular protein cytokeratin, DAPI stains for the cell nucleus and anti-CD45 FITC is specific for leukocytes. An event is classified as a tumor cell when its morphological features are consistent with that of a tumor cell and it exhibits the phenotype EpCAM⁺, CK⁺, DAN⁺, and CD45⁻.

The CTC number will be counted and analyzed by operators and recorded directly. For further immunostaining on CTCs, anti-PD-L1 will be automated injecting into SelectChip for labeling CTC on MiSelect R System. The fluorescence images of each biomarkers on CTC will be taken and intensity of the biomarkers will be recorded for further analysis.

4. Statistical Analyses

All data were statistically analyzed with SPSS software (v19.0) and GraphPad Prism (v5.0). The distributions of continuous variables were described as median values and ranges. The Mann-Whitney U test and the Wilcoxon-signed ranks test were performed to evaluate the differences between groups, as appropriate. All P-values were two-sided. P-values of less than 0.05 indicated statistical significance.

Results

1. Prevalence of CTC in PB and MVB

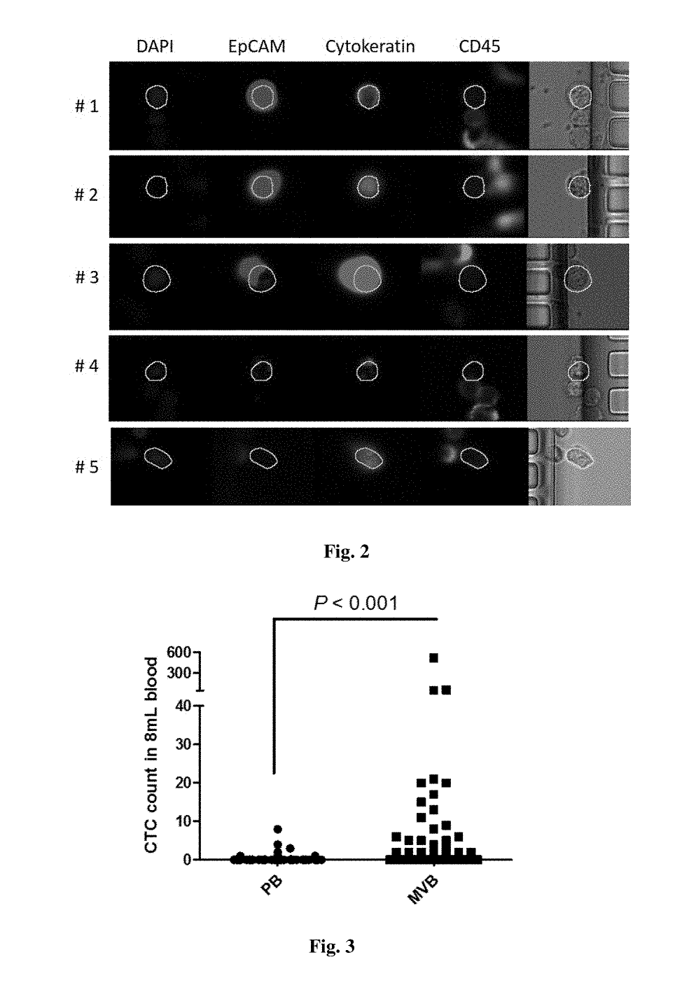

CTC was defined as a cell with intact nucleus, expressing EpCAM and cytokeratin, but absence of CD45 expression. Typical CTC images were demonstrated in FIG. 2. The EpCAM expression showed heterogeneity among CTCs even within the same CRC patient. CTCs were barely found in PB (mean, 0.17±0.89 per 8 ml of whole blood; range=0-8, n=116) but more abundant in MVB (mean, 7.1±48.1 per 8 ml of whole blood; range=0-515, n=116) (FIG. 3; P<0.001).

2. Distribution of CTC Counts and Detection Rates

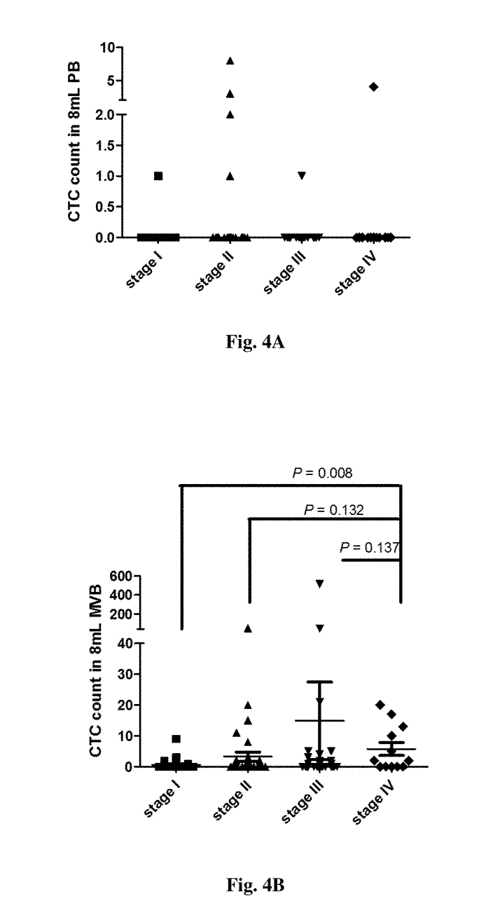

The CTC counts for CRC patients are presented in Table 2 below and in FIG. 4. CTC count ranged from 0 to 8 in non-metastatic CRC and 0 to 4 in metastatic CRC in PB; In MVB, CTC count ranged from 0 to 515 in non-metastatic CRC and 0 to 20 in metastatic CRC. The overall detection rate of CTC is 6% and 40.5% in PB and MVB, respectively. Within each subgroup, the detection rate increased with the severity of the subgroup's condition. In MVB, CTC detection rate was 20.8%, 42.1%, 45.2% and 58.3% for stage I, II, III, and IV respectively. Besides, the amount of CTC was significantly more abundant in late stages than early stages (FIG. 4).

TABLE 2
CTC count in various stages of PB and MVB

| | No. of | | Range of |
|---|---|---|---|
| | cases | | CTC number in |
| | N = 116 | | 8 ml |
| | | Detection rate | |
| | | in MVB % | |
| Stage I | 24 | 20.8% (5/24) | 0-9 |
| Stage II | 38 | 42.1% (16/38) | 0-20 |
| Stage III | 42 | 45.2% (19/42) | 0-515 |
| Stage IV | 12 | 58.3% (7/12) | 0-20 |
| | | P-value = | |
| | | 0.0298 | |
| | | Detection rate | |
| | | in PB % | |
| Stage I | 24 | 4.2% (1/24) | 0-1 |
| Stage II | 38 | 10.5% (4/38) | 0-8 |
| Stage III | 42 | 2.4% (1/42) | 0-1 |
| Stage IV | 12 | 16.6% (2/12) | 0-4 |
| | | P-value = | |
| | | 0.7053 | |

3. Relationships Between CTC Number and Clinicopathological Characteristics

We explored the bivariate relationship between CTC numbers (present or absent) versus various clinical and pathological parameters. The results are shown in Table 3 below. The clinical staging (TNM) positively correlated to CTC number in MVB (Table 2) and pre-operative serum CEA level and tumor invasion depth (pT) positively correlated to CTC levels in MVB. No association was noted between CTC numbers and presence of liver or lung metastases, primary CRC differentiation, histology, nodal status (N), lymphatic/venous invasion/perineural invasion, inflammatory change around carcinoma, invasion pattern of cancer tissue and pre-operative serum CA-19-9 level.

TABLE 3
Correlation between clinicopathological parameter and
the present of CTCs

| Clinicopathological | | | |
|---|---|---|---|
| variables | CTC present % | CTC absent % | P value |
| T stage | | | 0.018 |
| T1/T2 | 17% (6/34) | 83% (28/34) | |
| T3/T4 | 48% (41/84) | 52% (43/84) | |
| N stage | | | 0.13 |
| N0 | 34% (22/65) | 66% (43/65) | |
| N1 | 49% (25/51) | 51% (26/51) | |
| CEA | | | 0.049 |
| ≥5 mg/mL | 53% (23/44) | 47% (21/44) | |
| <5 mg/mL | 33% (23/71) | 67% (48/71) | |
| CA-199 | | | 0.33 |
| ≥37 mg/mL | 50% (11/22) | 50% (11/22) | |
| <37 mg/mL | 37% (35/94) | 63% (49/94) | |

4. PD-L1 Biomarker Assessment on CTCs in CRC Patients

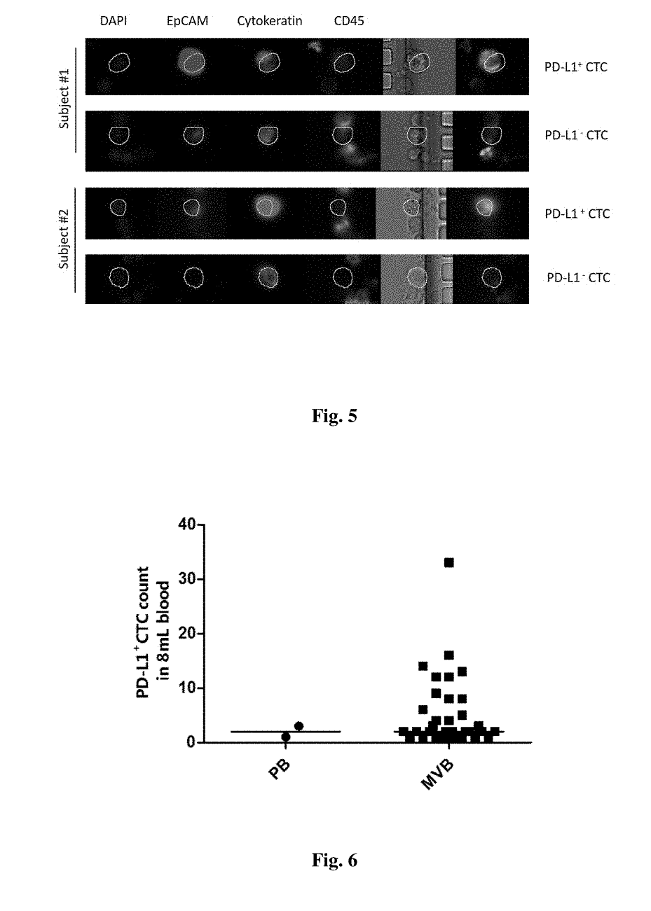

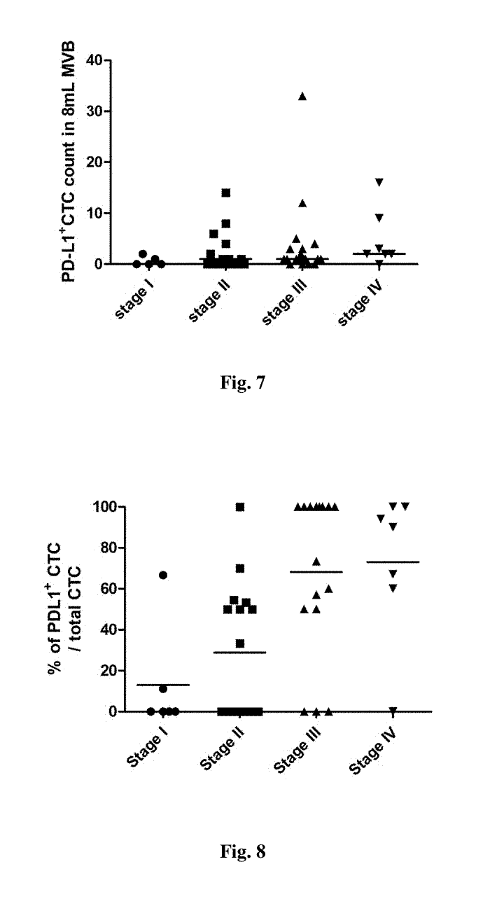

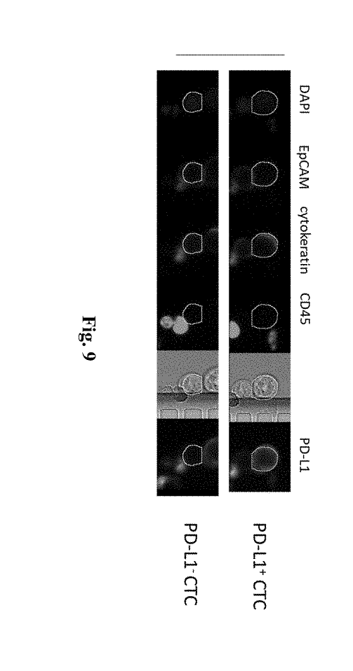

CTCs isolated from PB and MVB were examined for PD-L1 protein expression. The PD-L1 biomarker expression showed heterogeneity among isolated CTCs between patients and within the same blood sample (FIG. 5). PD-L1 status on CTC in PB was evaluated in 8 patients with detectable CTC (see Table 4 below). PD-L1 status on CTC in MVB was evaluated in 47 patients with detectable CTC. Among these 47 patients, 31 (65.9%) showed a subpopulation of PD-L1⁺ CTCs (see FIG. 6 and Table 5 below). The number of PD-L1⁺ CTCs varied from 1 to 33 (median=3) and the fraction of PD-L1⁺ CTCs ranged from 16 to 100% of the whole number of detectable CTCs. The PD-L1⁺ CTC number gradually increased with stages (FIG. 7) and the frequency of PD-L1⁺ CTCs among CTCs also increased with stages (FIG. 8).

TABLE 4
Number of PD-L1⁺ CTC in PB of various stages

| Patients | | | PD-L1⁺CTC |
|---|---|---|---|
| number | Stage | CTC number | number |
| 0031 | I | 1 | 0 |
| 0009 | II | 1 | 0 |
| 0078 | II | 1 | 0 |
| 0090 | II | 2 | 1 |
| 0113 | II | 3 | 0 |
| 0057 | III | 1 | 0 |
| 0004 | IV | 1 | 0 |
| 0075 | IV | 4 | 3 |

TABLE 5
Number of PD-L1⁺ CTC in MVB of various stages

| Patients | | | PD-L1⁺CTC |
|---|---|---|---|
| number | Stage | CTC number | number |
| 0011 | I | 1 | 0 |
| 0021 | I | 3 | 2 |
| 0031 | I | 1 | 0 |
| 0123 | I | 2 | 0 |
| 0129 | I | 9 | 1 |
| 0001 | II | 5 | 0 |
| 0009 | II | 2 | 2 |
| 0016 | II | 1 | 0 |
| 0037 | II | 2 | 1 |
| 0046 | II | 2 | 1 |
| 0051 | II | 2 | 0 |
| 0055 | II | 8 | 4 |
| 0060 | II | 1 | 0 |
| 0089 | II | 1 | 0 |
| 0091 | II | 1 | 0 |
| 0094 | II | 11 | 6 |
| 0096 | II | 2 | 0 |
| 0097 | II | 1 | 0 |
| 0103 | II | 15 | 8 |
| 0118 | II | 3 | 1 |
| 0139 | II | 20 | 14 |
| 0006 | III | 1 | 0 |
| 0012 | III | 4 | 4 |
| 0025 | III | 1 | 0 |
| 0030 | III | 1 | 1 |
| 0057 | III | 1 | 1 |
| 0069 | III | 5 | 5 |
| 0076 | III | 1 | 0 |
| 0077 | III | 3 | 3 |
| 0082 | III | 45 | 33 |
| 0084 | III | 21 | 12 |
| 0085 | III | 5 | 3 |
| 0086 | III | 2 | 1 |
| 0098 | III | 2 | 1 |
| 0100 | III | 1 | 1 |
| 0102 | III | 1 | 1 |
| 0104 | III | 2 | 1 |
| 0106 | III | 1 | 1 |
| 0134 | III | 1 | 0 |
| 0141 | III | 6 | 2 |
| 0004 | IV | 5 | 0 |
| 0017 | IV | 17 | 16 |
| 0028 | IV | 13 | 2 |
| 0034 | IV | 5 | 3 |
| 0038 | IV | 2 | 2 |
| 0068 | IV | 2 | 2 |
| 0075 | IV | 10 | 9 |

5. Relationships Between PD-L1⁺ CTC Number and Clinicopathological Characteristics in CRC Patients

We explored the bivariate relationship between PD-L1⁺ CTC numbers (present or absent) versus various clinical and pathological parameters. The results are shown in Table 6 and Table 7 below. The clinical staging (TNM) positively correlated to PD-L1⁺ CTC number in MVB (Table 6) and pre-operative serum CEA level, tumor invasion depth (pT), nodal status (N), positively correlated to PD-L1⁺ CTC levels in MVB. Besides, the primary CRC lymphatic and venous invasion were correlated to PD-L1⁺ CTC levels in MVB (Table 7). No association was noted between PD-L1⁺ CTC numbers and primary CRC differentiation, perineural invasion, inflammatory change around carcinoma, invasion pattern of cancer tissue and pre-operative serum CA-19-9 level.

TABLE 6
Correlation between PD-L1(+) CTC presence and clinical stage

| | Detection rate of | PD-L1(+) rate in |
|---|---|---|
| | PD-L1(+) CTC | CTC detected patients |
| | N = 116 | N = 47 |
| Stage I | 8.3% (2/24) | 40.0% (2/5) |
| Stage II | 21.1% (8/38) | 50.0% (8/16) |
| Stage III | 35.7% (15/42) | 78.9% (15/19) |
| Stage IV | 50.0% (6/12) | 85.7% (6/7) |
| | P-value = 0.0017 | P-value = 0.0178 |

TABLE 7
Correlation between clinicopathological parameter and
the present of PD-L1⁺ CTCs

| | PD-L1(+) | | |
|---|---|---|---|
| | CTC | PD-L1(+) CTC | |
| Clinicopathological variables | present (%) | absent (%) | P value |
| T stage | | | 0.016 |
| T1/T2 | 6% (2/33) | 94% (31/33) | |
| T3/T4 | 48% (23/83) | 52% (60/83) | |
| N stage | | | 0.013 |
| N0 | 12% (8/65) | 88% (57/65) | |
| N1 | 33% (17/51) | 67% (34/51) | |
| CEA | | | 0.002 |
| ≥5 mg/mL | 36% (17/47) | 64% (30/47) | |
| <5 mg/mL | 10% (7/67) | 90% (7/67) | |
| CA-199 | | | 0.13 |
| ≥37 mg/mL | 33% (7/21) | 67% (14/21) | |
| <37 mg/mL | 17% (16/92) | 83% (76/92) | |
| Blood Vascular Invasion | | | 0.014 |
| (+) | 42% (11/26) | 58% (15/26) | |
| (−) | 17% (14/84) | 83% (70/84) | |
| Lymphatic Vascular Invasion | | | 0.0042 |
| (+) | 36% (11/30) | 64% (19/30) | |
| (−) | 18% (14/80) | 82% (66/80) | |

6. PD-L1 Biomarker Assessment on CTCs in Other Cancer Patients

The overall detection rate of CTC is 31%, 55% and 40% in patients with head and neck squamous-cell carcinoma (HNSCC), hepatocellular carcinoma (HCC) and uterine cancer (UC), respectively (see Table 8 below). CTCs isolated from HNSCC, HCC and UC patients were examined for PD-L1 protein expression. The PD-L1 biomarker expression showed heterogeneity among isolated CTCs between patients and within the same blood sample (FIG. 9). PD-L1 status on CTC was evaluated in patients with detectable CTCs. Among these patients, 100% of HNSCC, 50% of HCC and 100% of UC showed a subpopulation of PD-L1⁺ CTCs (Table 8). The presence of PD-L1(+) CTC significantly correlates with disease progression in HNSCC patients (see Table 9 below).

TABLE 8
CTC and PD-L1(+) CTC detection rate in advanced cancers

| | | | PD-L1 positive |
|---|---|---|---|
| Cancer | CTC | PD-L1(+) rate in CTC | rate in CTC |
| type | detection rate % | detected patients % | (Range) |
| HNSCC | 31% (8/26) | 100% (8/8) | 12.5-100% |
| N = 26 | | | |
| HCC | 55% (4/9) | 50% (2/4) | 50-100% |
| N = 9 | | | |
| UC | 40% (2/5) | 100% (2/2) | 100% |
| N = 5 | | | |
| RCC | 0/2 | 0% | N.A. |
| N = 2 | | | |

TABLE 9
Correlation between CTC or PD-L1(+) CTC presence and
disease progression

| | CTC/PD-L1(+) CTC | | |
|---|---|---|---|
| | | | P- |
| Clinical outcome | present | absent | value |
| Progression disease | 7 | 4 | 0.034 |
| Complete response/ | 0 | 6 | |
| Partial response/ | | | |
| Stable disease | | | |

### REFERENCES

1. Pantel, K., C. Alix-Panabieres, and S. Riethdorf, Nat Rev Clin Oncol, 2009. 6(6): p. 339-51.
2. Cohen, S. J., et al., J Clin Oncol, 2008. 26(19): p. 3213-21.
3. Thorsteinsson, M., G. Soletormos, and P. Jess, Anticancer Res, 2011. 31(2): p. 613-7.
4. Sastre, J., et al., Ann Oncol, 2008. 19(5): p. 935-8.
5. Nicolazzo, C. et al., Sci Rep 2016 Aug. 24; 6:31726.

What is claimed is:

1. A method for identifying a subject at risk of developing a recurrent or metastatic cancer, comprising detecting PD-L1⁺ circulating tumor cells in a blood sample, a tissue fluid sample or a specimen of the subject, wherein the presence of one or more PD-L1⁺ circulating tumor cells indicates that the subject is at risk of developing a recurrent or metastatic cancer, and wherein the subject has a gastrointestinal cancer or head and neck squamous-cell carcinoma and the blood sample, the tissue fluid sample or the specimen is derived from the subject before a therapy for, or during a surgery of curative resection of the gastrointestinal cancer or head and neck squamous-cell carcinoma.

2. A method for treating a cancer comprising:
identifying a subject having PD-L1⁺ circulating tumor cells by detecting PD-L1⁺ circulating tumor cells in a blood sample, a tissue fluid sample or a specimen of the subject, wherein the subject has a gastrointestinal cancer or head and neck squamous-cell carcinoma and the blood sample, the tissue fluid sample or the specimen is derived from the subject before a therapy for, or during a surgery of curative resection of the gastrointestinal cancer or head and neck squamous-cell carcinoma; and
administering a treatment to the subject.

3. The method of claim 2, wherein the cancer is a recurrent or metastatic cancer.

4. The method of claim 2, wherein the treatment includes an immunotherapy.

5. The method of claim 3, wherein the treatment includes an immunotherapy.

6. The method of claim 1, wherein the cancer is a gastrointestinal cancer.

7. The method of claim 2, wherein the cancer is a gastrointestinal cancer.

8. The method of claim 6, wherein the gastrointestinal cancer is a colorectal cancer.

9. The method of claim 7, wherein the gastrointestinal cancer is a colorectal cancer.

10. The method of claim 6, wherein the blood sample is a mesenteric venous blood sample.

11. The method of claim 7, wherein the blood sample is a mesenteric venous blood sample.

12. A method for predicting prognosis of a patient having gastrointestinal cancer or head and neck squamous-cell carcinoma, comprising detecting PD-L1⁺ circulating tumor cells in a blood sample, a tissue fluid sample or a specimen of the patient, wherein the presence of one or more PD-L1⁺ circulating tumor cells indicates poor prognosis of the patient, and wherein the tissue fluid sample or the specimen is derived from the patient before a therapy for, or during a surgery of curative resection of the gastrointestinal cancer or head and neck squamous-cell carcinoma.

13. The method of claim 12, wherein the patient has a gastrointestinal cancer.

14. The method of claim 13, wherein the gastrointestinal cancer is a colorectal cancer.

15. The method of claim 13, wherein the blood sample is a mesenteric venous blood sample.
